(19) United States
(12) Patent Application Publication
(10) Pub. No.: US 2026/0202411 A1
(43) Pub. Date: Jul. 16, 2026

(54) METHODS FOR THE DETECTION AND TREATMENT OF LUNG CANCER

(71) Applicant: Board of Regents, The University of Texas System, Austin, TX (US)

(72) Inventors: Samir HANASH, Houston, TX (US); Johannes Francois FAHRMANN, Houston, TX (US); Ziding FENG, Houston, TX (US); Ehsan IRAJIZAD, Houston, TX (US)

(21) Appl. No.: 19/565,055

(22) Filed: Mar. 12, 2026

Related U.S. Application Data
(63) Continuation of application No. PCT/US2024/046877, filed on Sep. 16, 2024.
(60) Provisional application No. 63/584,680, filed on Sep. 22, 2023.

(51) Int. Cl. G01N 33/5752; G01N 33/575; G01N 33/68

(52) U.S. Cl. CPC G01N 33/5752; G01N 33/57585; G01N 33/6848; G01N 33/6884; G01N 2333/4725; G01N 2333/4742; G01N 2333/785

(57) ABSTRACT

Provided are methods for improving the lead time prior to diagnosis of lung cancer for a patient.

This application is a bypass continuation of International Application No. PCT/US2024/046877, filed Sep. 16, 2024, which claims the benefit of priority of U.S. Provisional Application No. 63/584,680, filed Sep. 22, 2023, the contents of which are incorporated by reference as if written herein in their entirety.

This invention was made with government support under CA200468, CA194733, CA213285, and CA086368 awarded by the National Institutes of Health. The government has certain rights in the invention.

Lung cancer is the most prevalent cancer in the United States, with a five-year survival rate of less than 15%. Recently, therapies for lung cancer have begun to transition from a limited selection of radiation, folate metabolism, platinum-based drugs, and taxane-based drugs to more targeted treatments that require histological characterization of the tumor and/or the presence or absence of key biomarker or therapeutic target proteins.

Data from the National Lung Screening Trial (NLST) suggests that yearly screening of high-risk current and ex-smokers with thoracic low-dose computed tomography (LDCT) has been shown to reduce mortality due to lung cancer by 20%. In 2021, the United States Preventive Service Task Force (USPSTF) expanded the eligibility for LDCT screening and now recommends annual screening for lung cancer with LDCT for adults aged 50-80 years who have a smoking history greater than 20 pack-years and either currently smoke or have quit within the past 15 years. However, there are several negative aspects associated with CT screening in terms of morbidity, including over-diagnosis, false positives, over-treatment, and financial costs.

There is an abundance of literature on lung cancer risk prediction on the potential benefit of supplementing the USPSTF screening criteria with a risk-based model when identifying subjects for CT-screening. For instance, recently it was estimated that 20% of additional lung cancer deaths could be avoided by using a screening criterion based on individual risk assessment. The information required to utilize risk-prediction tools could be readily ascertained by a general practitioner—or potentially self-assessed using an online risk-calculator—making future lung cancer screening programs likely to implement such tools when assessing screening eligibility.

One such tool would be an individual-level risk-based screening criteria that accurately estimates the risk of lung cancer within the near future (e.g., 1-3 years) for a given subject. Several risk prediction models have been published that rely on demographic data (age, sex, etc.) and risk factor data from questionnaires, such as PLCOₘ₂₀₁₂ and the Liverpool Lung Project (LLP). Elevated levels of protein biomarkers have also been found to serve as useful predictors of the risk of developing lung cancer. A novel blood-based four-marker protein panel comprising or consisting of pro-surfactant protein B (pro-SFTPB), Mucin 16 (CA125), carcinoembryonic antigen (CEA), and cytokeratin-19 fragment (CYFRA21-1) is described in U.S. Ser. No. 16/484,177, the contents of which are hereby incorporated by reference in their entirety. The use of this panel has been found to significantly improve lung cancer risk assessment compared to former and current USPSFT criteria for lung cancer screening.

Accordingly, a need exists for a method or test to improve the lead time prior to diagnosis of lung cancer. The use of a longitudinal screening method, comprising serial testing of the four-marker protein panel comprising or consisting of pro-surfactant protein B (pro-SFTPB), Mucin 16 (CA125), carcinoembryonic antigen (CEA), and cytokeratin-19 fragment (CYFRA21-1) in conjunction with a parametric empirical Bayes (PEB) algorithm, has been found to provide improved performance over a single time measurement.

### SUMMARY

Provided herein is a method of determining the risk of a patient for having a lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as being at risk for lung cancer or not being at risk for lung cancer by comparing the model score to a pre-defined parameter.

Also provided herein is a method of improving lead time prior to diagnosis of a patient having lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as being at risk for lung cancer or not being at risk for lung cancer by comparing the model score to a pre-defined parameter.

Also provided herein is a method, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from a patient asymptomatic for lung cancer at each time point;
identifying the patient as being at risk for lung cancer by comparing the model score to a pre-defined parameter; and
administering a computerized tomography (CT) scan to the patient identified as being at risk for lung cancer.

Also provided herein is a method of identifying and treating an asymptomatic patient with a risk for increased lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point;
identifying the patient as being at risk for lung cancer by comparing the model score to a pre-defined parameter;
administering a computerized tomography (CT) scan to the patient identified as being at risk for lung cancer; and
surgically removing the cancerous tumors identified on the CT scan.

### BRIEF DESCRIPTION OF THE DRAWINGS

FIG. 1 depicts ROC curves evaluating the 4MP performance with parametric empirical Bayes (PEB) and single threshold (ST) approaches.

FIG. 2 depicts ROC curves evaluating the 4MP performance with parametric empirical Bayes (PEB) and single threshold (ST) approaches in early stage lung cancer.

FIG. 3 depicts ROC curves evaluating the 4MP performance with parametric empirical Bayes (PEB) and single threshold (ST) approaches in late stage lung cancer.

FIG. 4 depicts ROC curves evaluating the 4MP performance with parametric empirical Bayes (PEB) and single threshold (ST) approaches in high risk smoking strata.

FIG. 5 depicts ROC curves evaluating the 4MP performance with parametric empirical Bayes (PEB) and single threshold (ST) approaches in medium risk smoking strata.

FIG. 6 depicts ROC curves evaluating the 4MP performance with parametric empirical Bayes (PEB) and single threshold (ST) approaches in low risk smoking strata.

FIG. 7 depicts ROC curves evaluating the 4MP performance with parametric empirical Bayes (PEB) and single threshold (ST) approaches in adenocarcinoma lung cancer.

FIG. 8 depicts ROC curves evaluating the 4MP performance with parametric empirical Bayes (PEB) and single threshold (ST) approaches in squamous lung cancer.

FIG. 9 depicts ROC curves evaluating the 4MP performance with parametric empirical Bayes (PEB) and single threshold (ST) approaches in small cell lung cancer.

FIG. 10 depicts PEB and ST positivity at the 1.7% 6-year specificity threshold, wherein both methods produce positive results on the first biomarker measurement tested. N=222.

FIG. 11 depicts PEB and ST positivity at the 1.7% 6-year specificity threshold, wherein neither method produces a positive test result. N=27.

FIG. 12 depicts PEB and ST positivity at the 1.7% 6-year specificity threshold, wherein the first positive result is either not from the first biomarker measurement or is positive by PEB or ST, but not both. N=75.

FIG. 13 depicts PEB and ST positivity at the 1.0% 6-year specificity threshold, wherein both methods produce positive results on the first biomarker measurement tested. N=261.

FIG. 14 depicts the distribution of the durations (in years) by which the PEB and ST method have different positive signals at 1.0% 6-year specificity threshold. A) Histogram distribution of the two approaches. B) Individual time representations of positive results. “+” represents no detection by PEB/ST method.

FIG. 15 depicts PEB and ST positivity at the 1.0% 6-year specificity threshold, wherein the first positive result is either not from the first biomarker measurement or is positive by PEB or ST, but not both. N=51.

### DETAILED DESCRIPTION

Provided herein is a method of determining the risk of a patient for having a lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as being at risk for lung cancer or not being at risk for lung cancer by comparing the model score to a pre-defined parameter.

Also provided herein is a method of improving lead time prior to diagnosis of a patient having lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as being at risk for lung cancer or not being at risk for lung cancer by comparing the model score to a pre-defined parameter.

Also provided herein is a method, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from a patient asymptomatic for lung cancer at each time point;
identifying the patient as being at risk for lung cancer by comparing the model score to a pre-defined parameter; and
administering a computerized tomography (CT) scan to the patient identified as being at risk for lung cancer.

Also provided herein is a method of identifying and treating an asymptomatic patient with a risk for increased lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point;
identifying the patient as being at risk for lung cancer by comparing the model score to a pre-defined parameter;
administering a computerized tomography (CT) scan to the patient identified as being at risk for lung cancer; and
surgically removing the cancerous tumors identified on the CT scan.

In some embodiments, the model score is calculated with the equation:

$$Model Score = \frac{Y - [ μ \times ( 1 - {B}_{n} ) + {\overline{X}}_{i} \times {B}_{n} ]}{\sqrt{V \times ( 1 - {B}_{1} \times {B}_{n} )}}$$

wherein:
Y is the biomarker score of the most recent time point and is calculated with the equation

$$0.473 * log [ CA 125 ] + 0.6531 * log [ CEA ] + 0.2612 * log [ CYFRA 21 - 1 ] + 0.9238 * log [ Pro - SFTPB ] ;$$

μ is about 7.07;
V is about 0.2672;
B₁ is about 0.767;
Bₙ is calculated with the equation (n*B₁)/(n*B₁+(1−B₁)), wherein n is the total number of tests performed on the patient; and
X̅ᵢ is the average of Y scores calculated from biomarkers collected at time points prior to the most recent time point.

In some embodiments, the pre-defined parameter is about 0.63.

In some embodiments, the pre-defined parameter is about 0.45.

In some embodiments, a model score greater than the pre-defined parameter is considered a positive test.

In some embodiments, a model score less than the pre-defined parameter is considered a negative test.

In some embodiments, the biomarker scores are collected at between two and five time points.

In some embodiments, the biomarker scores are collected at two time points.

In some embodiments, the biomarker scores are collected at three time points.

In some embodiments, the biomarker scores are collected at four time points. In some embodiments, the biomarker scores are collected at five time points.

In some embodiments, the individual is asymptomatic.

In some embodiments, the levels of biomarkers CEA, CA125, CYFRA21-1, and Pro-SFTPB in a biological sample obtained from the individual are determined by an immunoassay.

In some embodiments, each of the biomarkers CEA, CA125, CYFRA21-1, and Pro-SFTPB in a biological sample obtained from the individual generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the lung cancer is early stage (e.g., stage I or II).

In some embodiments, the lung cancer is advanced stage (e.g., stage III or IV).

In some embodiments, the individual has a smoking history of >10 pack years.

In some embodiments, the individual is between the age of 50 and 80 years.

In some embodiments, the AUC of the method is greater than the AUC for a different biomarker, biomarkers, panel, assay, algorithm, model, or any combination thereof.

In some embodiments, the AUC is greater than 0.81.

In some embodiments, the AUC is between 0.83 and 0.89.

In some embodiments, the AUC is about 0.86.

In some embodiments, the sensitivity and specificity values at a ≥1.7%/6-year risk threshold of the method are greater than the sensitivity and specificity values for a different biomarker, biomarkers, panel, assay, algorithm, model or any combination thereof.

In some embodiments, the sensitivity is greater than 0.81 and the specificity is about 0.63.

In some embodiments, the sensitivity is between 0.84 and 0.98.

In some embodiments, the sensitivity is about 0.91.

In some embodiments, the sensitivity and specificity values at a ≥1.0%/6-year risk threshold of the method are greater than the sensitivity and specificity values for a different biomarker, biomarkers, panel, assay, algorithm, model or any combination thereof.

In some embodiments, the sensitivity is greater than 0.91 and the specificity is about 0.45.

In some embodiments, the sensitivity is between 0.94 and 0.99.

In some embodiments, the sensitivity is about 0.96.

In some embodiments, the lead time to diagnosis of a patient having lung cancer is greater than the lead time to diagnosis for a different biomarker, biomarkers, panel, assay, algorithm, model or any combination thereof.

In some embodiments, the lead time to diagnosis is greater than 1.03 years.

In some embodiments, the lead time to diagnosis is between 1.26 and 2.70 years.

In some embodiments, the patient is subsequently administered further lung cancer screening or treatment.

In some embodiments, the screening is chosen from endoscopic ultrasound, magnetic resonance imaging (MRI), and computed topography (CT) scans.

In some embodiments, the screening is performed annually.

In some embodiments, the screening is performed semi-annually.

In some embodiments, the treatment is chosen from surgery, chemotherapy, immunotherapy, radiation therapy, targeted therapy, or a combination thereof.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

As used herein, “lung cancer” refers to a malignant neoplasm of the lung characterized by the abnormal proliferation of cells, in which the growth of the cells exceeds and is uncoordinated with that of the normal tissues around it. In some embodiments, lung cancer may vary in severity, represented by stages I through IV. In some embodiments, lung cancer may be in an early stage (e.g., stage I or II), or it may be advanced (e.g., stage III or IV).

As used herein, the terms “subject” or “patient” refer to a mammal, preferably a human, for whom a classification as lung cancer-positive or lung cancer-negative is desired, and for whom further treatment can be provided.

As used herein, “healthy” refers to an individual in whom no evidence of lung cancer is found, i.e., the individual does not have lung cancer. Such an individual may be classified as “lung cancer-negative” or as having healthy lungs, or normal, non-compromised lung function. A healthy patient or subject has no symptoms of lung cancer, but may have benign lung nodules or masses, i.e., a combination of adenomas and cysts, or a non-cancerous lung condition or conditions, such as chronic obstructive pulmonary disease (COPD). In some embodiments, a healthy patient or subject may be used as a comparison to diseased or suspected diseased samples for determination of lung cancer in a patient or a group of patients.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery. Pharmacological substances as used herein may include, but are not limited to, anticancer drugs including chemotherapeutics, polyamine inhibitors, hormone therapies, and targeted therapies. Examples of chemotherapeutics for lung cancer include paclitaxel/Taxol (e.g. albumin bound paclitaxel or nab-paclitaxel, trade name Abraxane®), erlotinib (Tarceva® and others), afatinib (Gilotrif®), gefitinib (Iressa®), bevacizumab (Avastin®), gemcitabine (Gemzar®), crizotinib (Xalkori®), ceritinib (Zykadia®), cisplatin/Platinol, carboplatin (Paraplatin®), docetaxel (Taxotere®), pemetrexed (Alimta®), and vinorelbine (Navelbine®); as well as combination regimens of chemotherapy including cisplatin+paclitaxel, TIP (paclitaxel/Taxol, ifosfamide, and cisplatin/Platinol), VeIP (vinblastine, ifosfamide, and cisplatin/Platinol), VIP (etoposide/VP-16, ifosfamide, and cisplatin/Platinol), VAC (vincristine, dactinomycin, and cyclophosphamide), and PEB (cisplatin/Platinol, etoposide, and bleomycin). The terms “pharmacological substance” and “anticancer therapy” may also include substances used in immunotherapy, such as checkpoint inhibitors. Treatment may include a multiplicity of pharmacological substances, or a multiplicity of treatment methods, including, but not limited to, surgery and chemotherapy.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc.), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “elevated” refers to a biomarker level or model score in a given subject that is greater relative to the same biomarker level or model score in a given set of healthy patients or subjects. In some embodiments, an elevated PLCOₘ₂₀₁₂ model score is 0.00948 or greater. In some embodiments, an elevated PLCOₘ₂₀₁₂ model score is 0.016082 or greater.

As used herein, the term “hazard ratio” refers to a measure of how often a particular event happens in one group compared to how often it happens in another group, over time. Hazard ratios are often used in clinical trials to measure survival at any point in time in a group of patients who have been given a specific treatment compared to a control group given another treatment or a placebo. Hazard is defined as the slope of the survival curve-a measure of how rapidly subjects are dying. A hazard ratio of one means that there is no difference in survival between the two groups. A hazard ratio of greater than one or less than one means that survival was better in one of the groups. If the hazard ratio is 2.0, then the rate of deaths in one treatment group is twice the rate in the other group.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for lung cancer positive.

As used herein, the term “biomarker score” refers to a numerical score for a given biomarker or set of biomarkers measured in a sample from a subject. The biomarker score is calculated by normalizing or weighting the measured levels using fixed coefficients as prescribed by the statistical method for a given biomarker panel. Biomarker scores are used as components in calculating a model score for the subject.

As used herein, the “use” of markers for diagnosing lung cancer refers to quantification of the levels or amounts in a biological sample of one or more markers described herein. Quantification may be done using any known methods or techniques in the art or described herein. In some embodiments, markers may be used or combined together as a panel for statistical comparison to other samples.

As used herein, the term “classification” refers to the assignment of a subject as being at risk for lung cancer or not being at risk for lung cancer, based on the result of the biomarker score, risk score, or risk profile that is obtained for said subject.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for lung cancer-positive and lung cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “positive predictive value” refers to the proportion of positive results derived by a certain method that are truly positive.

As used herein, the term “disease progression” or “early disease progression” is defined as upgrading of Gleason score and/or increased tumor volume on surveillance biopsy within 18 months after start of active surveillance.

The phrase “therapeutically effective” is intended to qualify the amount of active ingredients used in the treatment of a disease or disorder or on the effecting of a clinical endpoint.

### LIST OF ABBREVIATIONS

4MP=four-marker protein panel (pro-surfactant protein B (pro-SFTPB), Mucin 16 (CA125), carcinoembryonic antigen (CEA), and cytokeratin-19 fragment (CYFRA21-1)); AUC=area under the curve; ROC=receiver operating characteristic.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: The PLCO Specimen Set

### The PLCO Cohort

The PLCO Cancer Screening Trial was a randomized multicenter trial in the United States which aimed at evaluating the impact of early detection procedures for prostate, lung, colorectal and ovarian cancer on disease-specific mortality. A biorepository was created for blood specimens that were annually collected from consented, intervention group participants. Reporting of cancer status was based on annual questionnaires. Medical records were obtained to document diagnostic follow-up and characteristics of any diagnosed lung cancers. The TNM stage and stage group were determined by the fifth edition of the American Joint Committee on Cancer's Cancer Staging Manual. Treatment data were abstracted from medical records for the 1-year period following diagnosis. PLCO participants were followed for an additional 13 years after the PLCO study ended for lung cancer incidence and 20 years for lung cancer death.

All deaths occurring during the trial were ascertained primarily through annual study update questionnaires. Participants who did not return the questionnaire were contacted by repeat mailing or telephone. To enhance the completeness of end point verification, the active follow-up was accompanied by periodic linkage to the National Death Index. Death certificates were obtained to confirm the death and to determine the provisional cause of death. As the underlying cause of death was not always accurately recorded on the death certificate, the PLCO trial used an end-point adjudication process to assign cause of death in a uniform and unbiased manner. All deaths with causes potentially related to cancer were reviewed by a death review committee with a nonvoting chair and three experience reviewers. Death reviewers were blinded to the trial group of the deceased participant. Lung cancer-specific deaths were defined as those with underlying cause of lung cancer or treatment for lung cancer.

All histologically confirmed lung cancers from the ever-smoked participants with at least two blood draws in the intervention arm that were diagnosed within six years of study entry and at least one biomarker measurement within 2 years of diagnosis (n=338 case participants) were selected for the current study. Non-case participants who have ever smoked and had at least two blood draws were randomly selected (n=2,432 non-cases). (Table 1). Participants with ≥10 PYs of smoking were chosen as the intended screening population (Table 2).

TABLE 1
Number of participants by number of serial biomarker measurements

| Number of | | | |
|---|---|---|---|
| Tests | Case Participants | Non-case Participants | Total Participants |
| 5 | 28 | 1,000 | 1,028 |
| 4 | 47 | 463 | 510 |
| 3 | 143 | 496 | 639 |
| 2 | 120 | 135 | 255 |
| Total | 338 | 2,094 | 2,432 |

TABLE 2
Number of participants by number of serial biomarker measurements
(≥10 PYs of smoking)

| Number of | | | |
|---|---|---|---|
| Tests | Case Participants | Non-case Participants | Total Participants |
| 5 | 27 | 782 | 809 |
| 4 | 44 | 377 | 421 |
| 3 | 139 | 403 | 542 |
| 2 | 114 | 112 | 226 |
| Total | 324 | 1,674 | 1,998 |

### Assaying the 4MP in the PLCO Specimen Set

Samples from all study participants for both training and testing, were sent on dry ice blinded to case-control status to the laboratory at MD Anderson Cancer Center, where they were kept below −80° C. until analysis. Concentrations for pro-SFTPB, CA125, CEA, and CYFRA21-1 were determined using bead-based immunoassays on the MAGPIX® instrument (Luminex Corporation, Austin TX). Samples were analyzed in batches of 36 samples in duplicates with matched cases and controls in the same batch in random order. Quality control procedures included 7 calibration standards, 2 Quality Control samples, and 1 blank sample run in duplicate in each batch. The coefficients of variation (CVs) within and between batches were 6.86% and 15.54% for CA125, 1.45% and 9.32% for CEA, 6.55% and 17.26% for pro-SFTPB, and 5.56% and 28.71% for CYFRA21-1, respectively. Biomarker scores for the 4MP were derived using fixed beta-coefficients from a previously developed logistic regression model. See U.S. Ser. No. 16/484,177. Coefficients of variation (CV) values for pro-SFTPB, CA125, CEA, and CYFRA21-1 in quality control samples were 22.2, 12.8, 10.8, and 22.6 percent, respectively.

### Statistical Analyses

A single threshold (ST) method, that compared only the current marker measurement against the identical threshold for everyone in the population, was considered versus a parametrical empirical Bayes (PEB) algorithm, which adjusts the biomarker threshold at each test to reflect participant history. Briefly, the PEB algorithm utilizes a simple model of biomarker values among non-cases from which the overall mean biomarker value (u) and variability of measurements within and across non-case participants are estimated. These parameters are used to center and scale biomarker measurements, with the current measurement compared against a weighted average of u and the participant's previous biomarker values. With increasing repeat measurements of the biomarker, the PEB reference level, and thus the test threshold, becomes increasingly individualized. In contrast, the ST method compares the current biomarker measurement against the non-case reference level u. In both methods, large deviations from the respective reference level indicate values unusually high in the non-case population.

For this study, the false-positive rate (FPR) was estimated at the screening level, defined as the proportion of positive results among all the screenings conducted in the control group. The screening-level specificity is defined as 1-FPR. The true-positive rate (TPR) or sensitivity at the patient level, which is defined as the proportion of lung cancer cases with at least 1 positive biomarker ‘test’, was estimated to be consistent with previous applications of this method. Thresholds that span the full range of false positive rates and plot an ROC curve of TPR vs FPR and calculate the area under the curve (AUC) were selected.

PEB parameters were estimated from non-case participants with ≥10 PY smoking history (Table 3).

TABLE 3
Population parameters estimated from individuals with 10
pack-years of smoking history with at least
2 biomarker measurements and no lung cancer diagnosis.

| | Calculated 4MP |
|---|---|
| Parameter | Value |
| μ | 7.07 |
| τ² | 0.205 |
| σ² | 0.0622 |
| ICC (B) | 0.767 |

To account for outcome-dependent sampling and multiple measurements per participant, 95% CI were calculated as percentiles of the sampling distribution estimated from 1,000 resamples using a stratified and clustered bootstrap method. Analyses were performed using R software version 4.2.0 (R Project for Statistical Computing). The population mean (μ), variance (V), and intraclass correlation (ICC, or B) were estimated by using a linear random-effects model. Specifically, Xᵢⱼ is equal to the 4MP biomarker score for person i at screen j=1 . . . n and follows the statistical model Xᵢⱼ|μᵢ~N(μᵢ, σ²) with the person means μᵢ varying in the population by μᵢ~N (μ̅, τ²). This model implies that a single (n=1) measurement has a population mean μ̅ variance:

$$V = {σ}^{2} + {τ}^{2}$$

$$and ICC$$

$$B = {τ}^{2} / ( {σ}^{2} + {τ}^{2} )$$

The ICC measures the degree of similarity in biomarker levels between individuals compared within individuals. The multilevel package of the R software was used to estimate B₁. At each screen, a PEB-deviation model score was calculated for each patient by comparing their current biomarker score for the 4MP (Y) to a function that includes the sample mean of the patient's n≥0 previous 4MP scores, denoted X̅ᵢ. The ICC of that sample mean (Bₙ) is calculated as

$${B}_{n} = ( n \times B ) / ( n \times B + ( 1 - B ) )$$

The PEB model score is calculated as

$$Model Score = \frac{Y - [ μ \times ( 1 - {B}_{n} ) + {\overline{X}}_{i} \times {B}_{n} ]}{\sqrt{V \times ( 1 - {B}_{1} \times {B}_{n} )}}$$

The ‘test’ is considered to be positive when the model score is greater than a pre-determined cutoff parameter, where the cutoff can be estimated empirically from training data or by using percentiles of the standard normal distribution.

### Predictive Performance of the PEB Model for Diagnosis of Lung Cancer

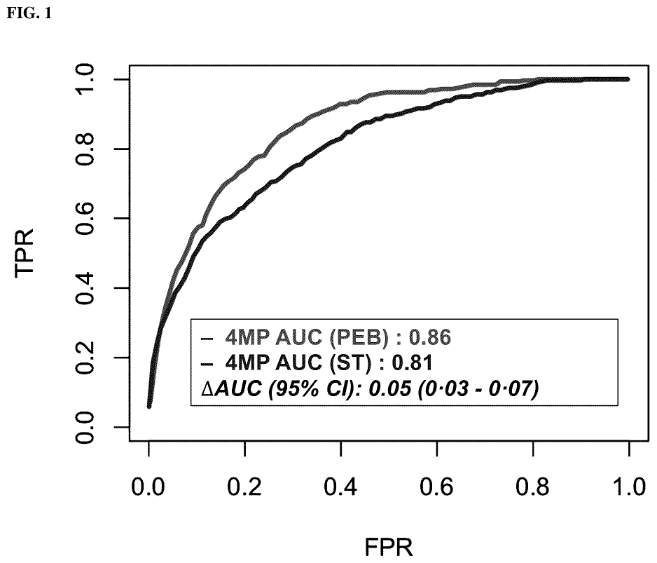

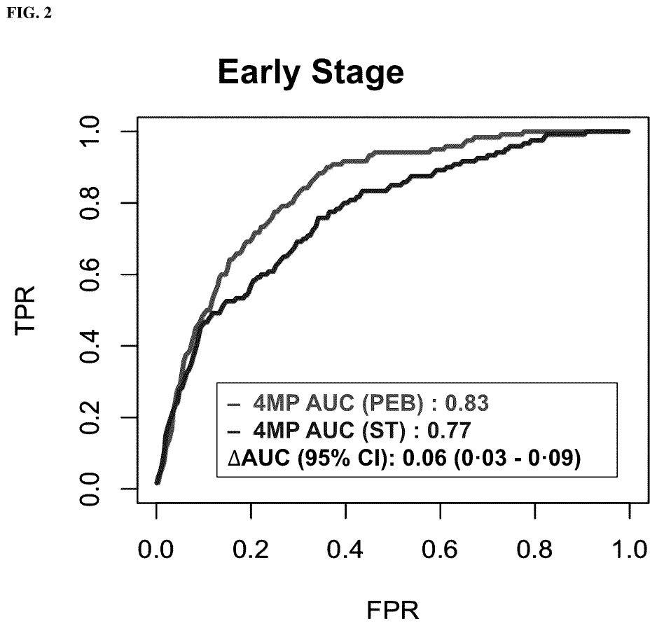

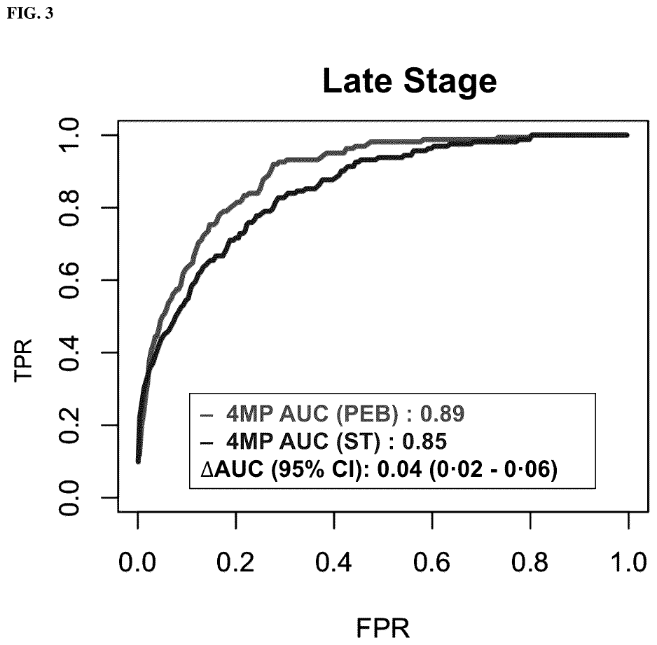

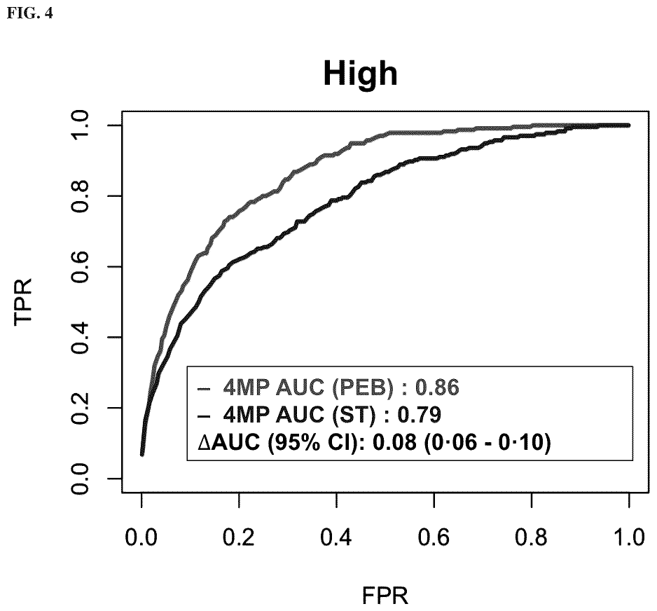

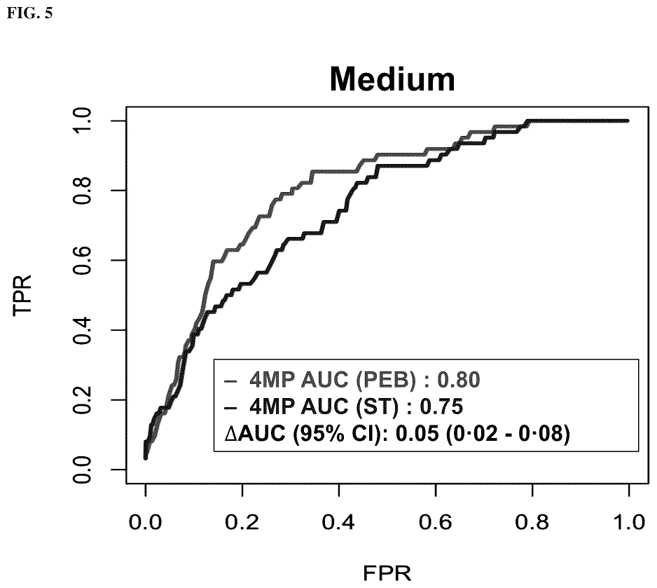

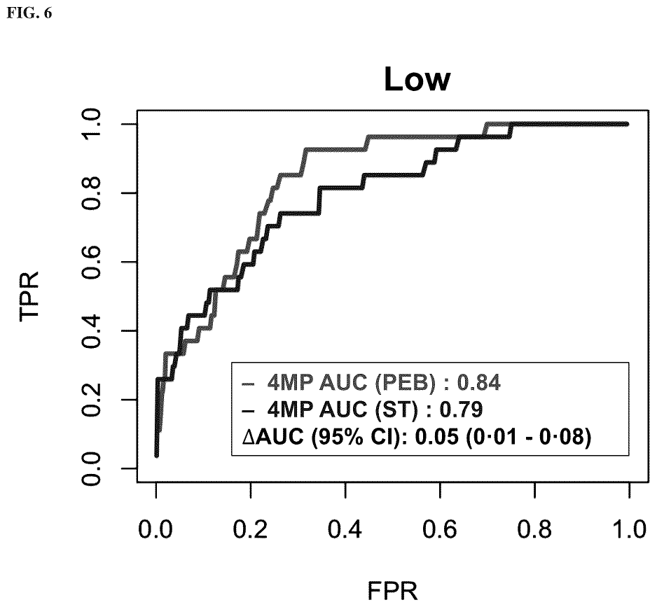

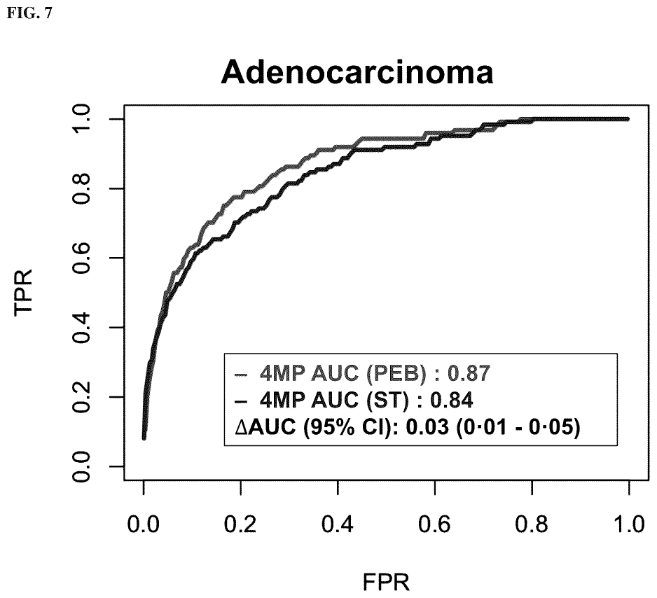

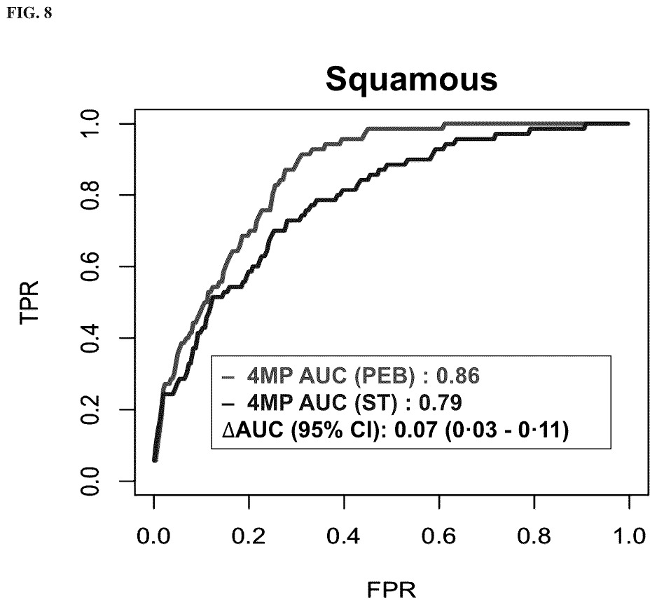

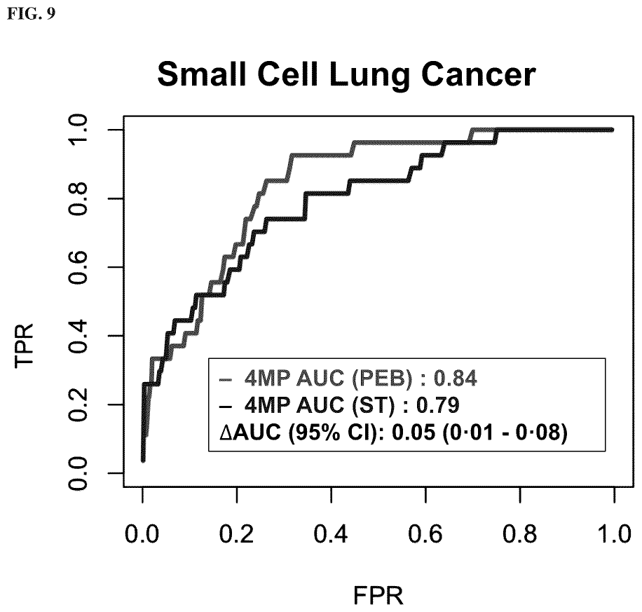

Amongst individuals with ≥10 PYs, the PEB algorithm, which considers repeated measurements of the 4MP, yielded an additional 5% improvement in area under the curve (AUC) compared to the ST approach (AUC_{PEB}:0.86 vs AUC_{ST}:0.81; P-value<0.05) (FIG. 1). Benefit of the PEB algorithm was observed among cases stratified into those that were clinically diagnosed with early-stage (I+II) or late-stage (III-IV) lung cancer with respective AUC improvements of 0.06 (AUC_{PEB}:0.83 vs AUC_{ST}:0.77; P-value<0.05) and 0.04 (AUC_{PEB}:0.89 vs AUC_{ST}:0.85; P-value<0.05) (FIGS. 2-9). Comparison of PEB versus ST among low-, medium-, and high-risk strata, are provided in FIGS. 4-6.

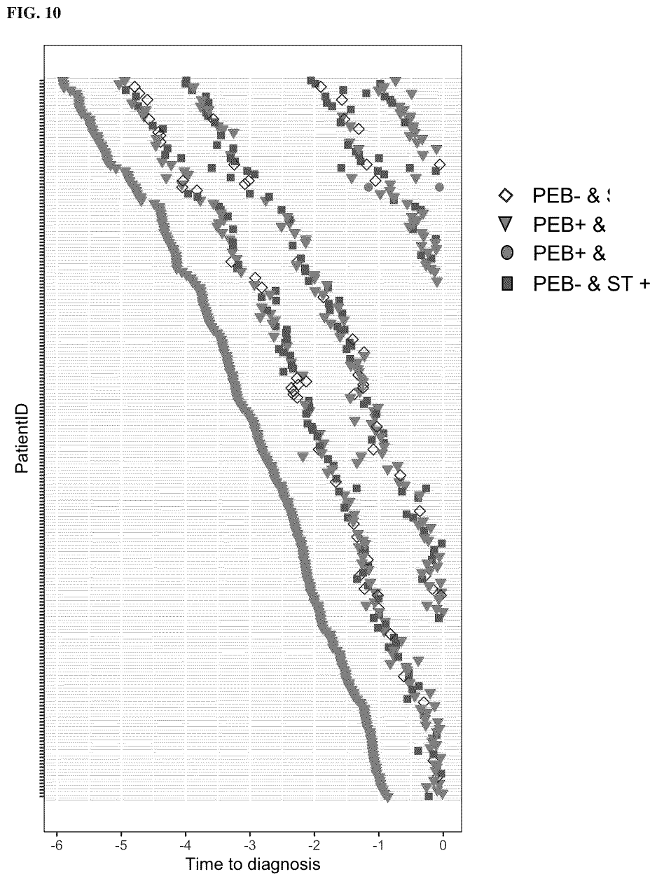

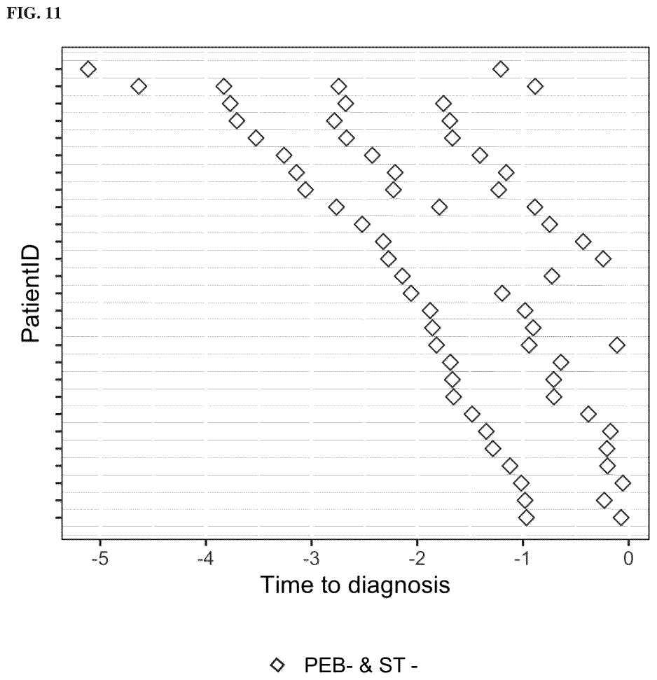

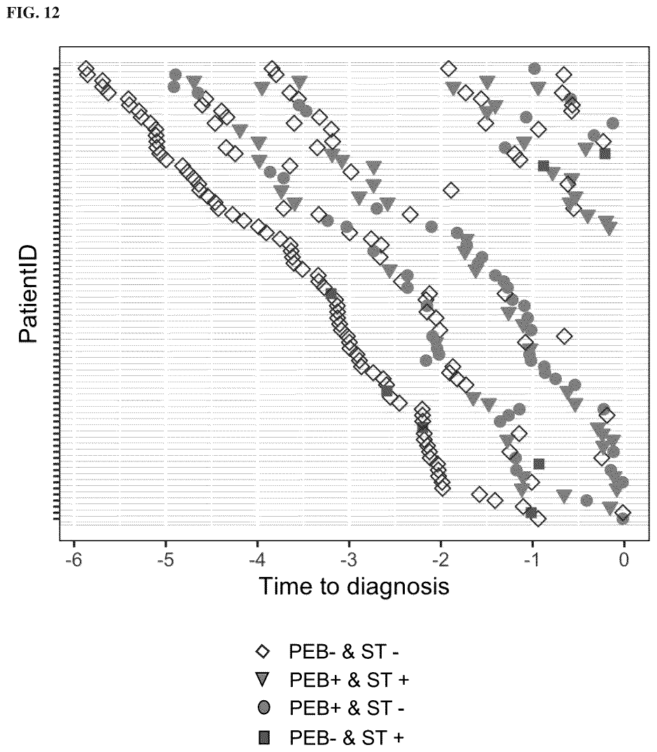

At a pre-established specificity threshold of 63.2%, which corresponds to the specificity of the 4MP at 1.7% 6-year risk threshold, PEB improved sensitivity by 9.8% (Sen_{PEB}:90.7% vs Sen_{ST}:80.9; P-value<0.05), which is equivalent to detecting 48.7% of the case individuals that were missed by ST (Table 4). Of the 324 cases with ≥10 PYs, 297 (92.6%) had a positive ‘test’ either by PEB or ST. 222 of the 297 (74.7%) had a positive PEB/ST ‘test’ result on the first biomarker measurement (FIGS. 10 and 11). Among the 75 cases that were initially negative ‘test’ on the first blood draw, 45 (60.0%) received an earlier positive ‘test’ based on the PEB algorithm compared to the ST approach with an average lead time of 1.21 (IQR: 0.63-2.07) (FIG. 12, Table 5).

TABLE 4
Sensitivity at pre-defined specificity for PEB and ST methods.

| | | 4MP | | |
|---|---|---|---|---|
| | | PEB | ST | |
| Criteria | Specificity | Sensitivity | Sensitivity | Delta Sensitivity |
| 1% 6-year risk¹ | 0.454 | 0.963 | 0.910 | 0.053 (0.022-0.083) |
| 1.7% 6-year risk² | 0.632 | 0.907 | 0.809 | 0.098 (0.074-0.125) |

¹Corresponding to USPSTF2021 criteria.
²Corresponding to USPSTF2013 criteria.

TABLE 5
Lead time (in years) of PEB over ST in different subgroups at
1.7% 6-year risk specificity threshold (63.2%).

| | | Lead time in years | |
|---|---|---|---|
| | Subgroups | (1^{st} quantile-3^{rd} quantile) | N |
| Risk groups | High risk | 1.58 (0.938-2.119) | 26 |
| | Medium risk | 1.58 (0.828-1.988) | 11 |
| | Low risk | 1.48 (0.129-2.676) | 3 |
| Subtypes | Adenocarcinoma | 1.46 (0.94-2.49) | 12 |
| | Squamous | 1.08 (0.779-1.689) | 14 |
| | Small Cell Cancer | 1.33 (0.732-2.168) | 7 |
| Stages | Early Stage | 1.11 (0.574-1.627) | 20 |
| | Late Stage | 1.45 (0.908-2.315) | 22 |

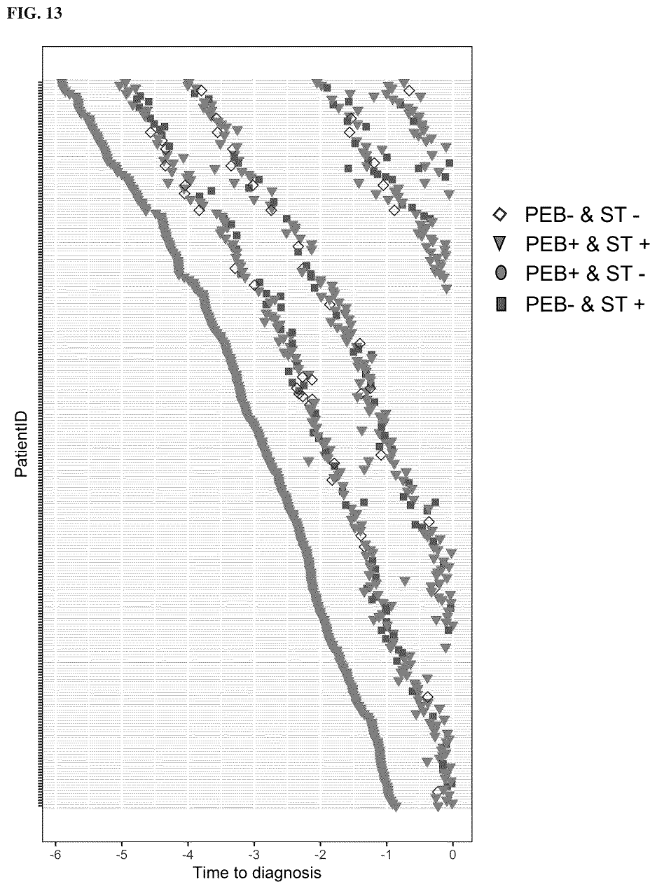

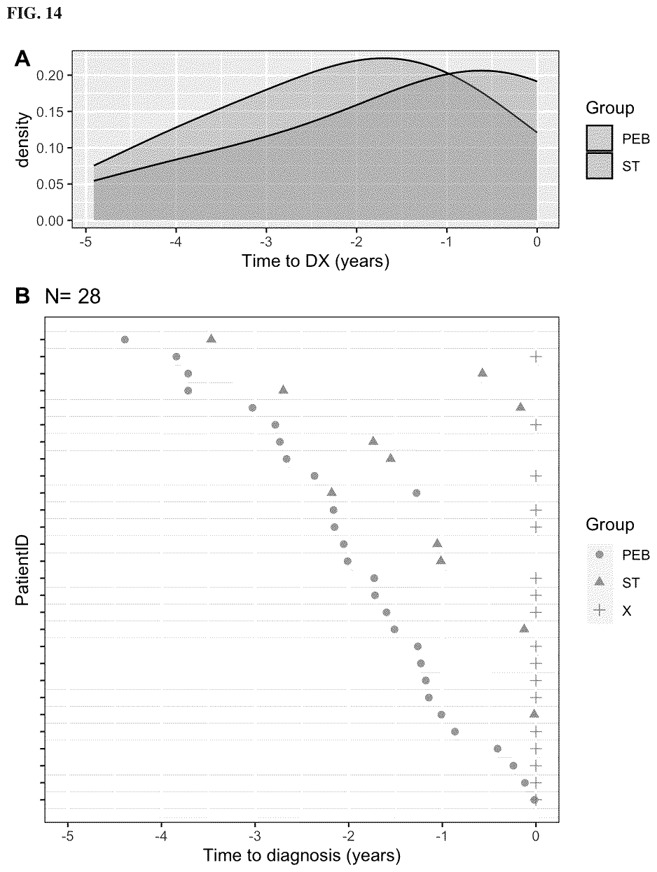

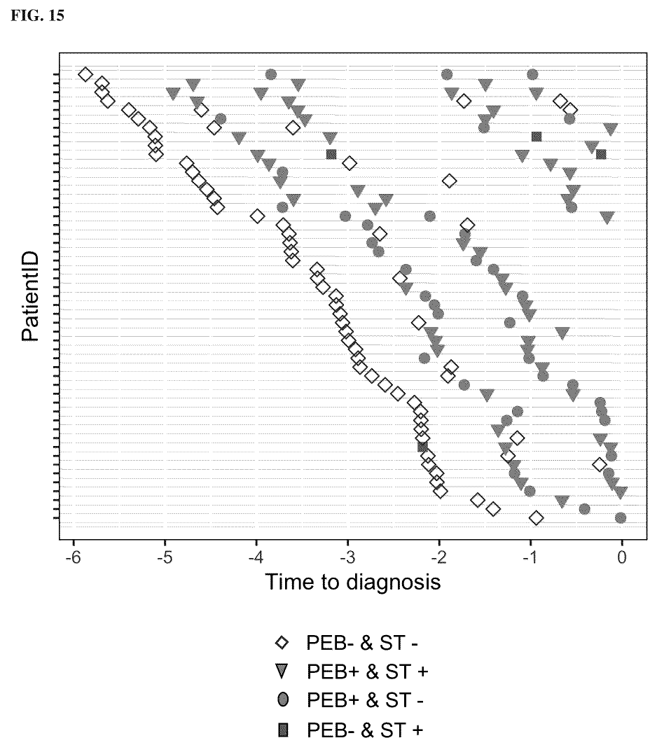

At a specificity threshold of 45.4%, which corresponds to 1% 6-year risk (corresponding to current USPSTF2021 screening guideline criteria), PEB improved sensitivity by 5.3% (Sen_{PEB}:96.3% vs Sen_{ST}:91.0; P-value<0.05) (Table 4). Of the 324 cases with ≥10 PYs, 312 (96.3%) had a positive ‘test’ either by PEB or ST. 261 of the 312 (83.6%) had a positive PEB/ST ‘test’ result on the first biomarker measurement (FIG. 13). Among the 51 cases that were initially ‘test’-negative on the first blood draw, 27 (52.9%) received an earlier positive ‘test’ based on the PEB algorithm compared to the ST approach with an average lead time of 1.37 (IQR: 0.97-1.83) (FIGS. 14 and 15; Table 6).

TABLE 6
Lead time (in years) of PEB over ST in different subgroups at
1.0% 6-year risk specificity threshold (45.4%)

| | | Lead time in years | |
|---|---|---|---|
| | Subgroups | (1^{st} quantile-3^{rd} quantile) | N |
| Risk groups | High risk | 1.62 (0.99-2.54) | 14 |
| | Medium risk | 0.41 (−1.01-1.63) | 11 |
| | Low risk | −2.78 (−4.18-−2.07) | 9 |
| Subtypes | Adenocarcinoma | 1.85 (1.11-2.93) | 14 |
| | Squamous | 1.54 (1.28-1.73) | 6 |
| | Small Cell Cancer | 1.23 (0.92-1.016) | 5 |
| Stages | Early Stage | 1.35 (0.96-1.72) | 21 |
| | Late Stage | 1.61 (1.04-2.31) | 10 |

At a pre-established specificity threshold of 45.4% (corresponding to a 1.0% 6-year risk of lung cancer), of the 28 individuals that had a positive signal from either PEB or ST, the PEB algorithm signaled a positive result in 17 individuals with lead time of 1.26 years (IQR: 0.87-2.15) before diagnosis while ST remained negative (Table 7). 6 of the 17 (35.3%) were non-eligible for LDCT screening based on USPSTF2021 criteria. Ten individuals that were positive based on ST with an average lead time of 1.03 years (IQR: 0.27-1.69) were positive based on PEB with an average lead time of 2.70 years (IQR: 2.02-3.54) (FIG. 14; Tables 7-9, 12).

At a pre-established specificity threshold of 63.2% (corresponding to a 1.7% 6-year risk of lung cancer), of the 50 individuals that had a positive signal from either PEB or ST, the PEB algorithm signaled a positive result that would trigger CT screening in 35 individuals that remained ST negative, at an average lead time of 1.19 years (interquartile range (IQR): 0.81-1.92) before diagnosis. Of those, 17 (48.6%) were non-eligible for LDCT screening based on USPSTF2013 eligibility criteria (Table 7). Ten individuals that were positive based on ST with an average lead time of 0.90 years (IQR: 0.46-1.48) were positive based on PEB with an average lead time of 3.35 years (IQR: 2.81-3.67) (Tables 7, 10-12).

TABLE 7
Lead time estimation at pre-defined specificity for PEB and ST
methods amongst individual with ≥10 PY smoking history.
1% 6-year risk
Improvement of PEB

| | ST remained | PEB signaled positive |
|---|---|---|
| | negative | earlier than ST |
| N | 17 | 10 |
| # of Non-USPSTF2021 eligible | 6 | 1 |
| Lead Time of PEB (Years, IQR) | 1.26 (0.87-2.15) | 2.70 (2.02-3.54) |
| Lead Time of ST (Years, IQR) | NA | 1.03 (0.27-1.69) |

Improvement of ST

| | PEB remained | ST signaled positive |
|---|---|---|
| | negative | earlier than PEB |
| N | 0 | 1 |
| # of Non-USPSTF2021 eligible | 0 | 0 |
| Lead Time of PEB (Years, IQR) | NA | 1.28 |
| Lead Time of ST (Years, IQR) | NA | 2.18 |

1.7% 6-year risk
Improvement of PEB

| | ST remained | PEB signaled positive |
|---|---|---|
| | negative | earlier than ST |
| N | 35 | 10 |
| # of Non-USPSTF2013 eligible | 17 | 1 |
| Lead Time of PEB (Years, IQR) | 1.19 (0.81-1.92) | 3.35 (2.81-3.67) |
| Lead Time of ST (Years, IQR) | NA | 0.90 (0.46-1.48) |

Improvement of ST

| | PEB remained | ST signaled positive |
|---|---|---|
| | negative | earlier than PEB |
| N | 3 | 2 |
| # of Non-USPSTF2013 eligible | 0 | 0 |
| Lead Time of PEB (Years, IQR) | NA | 0.45 (0.37-0.54) |
| Lead Time of ST (Years, IQR) | 1.02 (0.97-2.11) | 2.39 (2.30-2.49) |

N represents the number of case participants;
Abbreviations: IQR—interquartile range

TABLE 8
Lead time estimation of PEB and ST methods at a predefined 1.0%
6-year risk specificity threshold among different risk strata.
High risk strata
Improvement of PEB

| | ST remained | PEB signaled |
|---|---|---|
| | negative | earlier than ST |
| N | 5 | 9 |
| # of Non-USPSTF2013 eligible | 0 | 0 |
| Lead Time of PEB (Years, IQR) | 2.21 (1.23-2.65) | 3.03 (2.01-3.72) |
| Lead Time of ST (Years, IQR) | NA | 0.98 (0.41-2.70) |

Improvement of ST

| | PEB remained | ST signaled |
|---|---|---|
| | negative | earlier than PEB |
| N | 0 | 0 |
| # of Non-USPSTF2013 eligible | 0 | 0 |
| Lead Time of PEB (Years, IQR) | NA | NA |
| Lead Time of ST (Years, IQR) | NA | NA |

Medium risk strata
Improvement of PEB

| | ST remained | PEB signaled |
|---|---|---|
| | negative | earlier than ST |
| N | 2 | 2 |
| # of Non-USPSTF2013 eligible | 2 | 1 |
| Lead Time of PEB (Years, IQR) | 1.08 (0.55-1.62) | 3.13 (2.90-3.37) |
| Lead Time of ST (Years, IQR) | NA | 0.84 (0.48-1.20) |

Improvement of ST

| | PEB remained | ST signaled |
|---|---|---|
| | negative | earlier than PEB |
| N | 2 | 1 |
| # of Non-USPSTF2013 eligible | 2 | 0 |
| Lead Time of PEB (Years, IQR) | NA | 3.86 |
| Lead Time of ST (Years, IQR) | 1.50 (1.31-1.69) | 4.76 |

Low risk strata
Improvement of PEB

| | ST remained | PEB signaled |
|---|---|---|
| | negative | earlier than ST |
| N | 1 | 0 |
| # of Non-USPSTF2013 eligible | 1 | 0 |
| Lead Time of PEB (Years, IQR) | 1.16 | NA |
| Lead Time of ST (Years, IQR) | NA | NA |

Improvement of ST

| | PEB remained | ST signaled |
|---|---|---|
| | negative | earlier than PEB |
| N | 4 | 2 |
| # of Non-USPSTF2013 eligible | 4 | 2 |
| Lead Time of PEB (Years, IQR) | NA | 0.23 (0.18-0.28) |
| Lead Time of ST (Years, IQR) | 3.93 (3.47-4.29) | 2.86 (2.06-3.66) |

N represents the number of case participants;
Abbreviations: IQR—interquartile range

TABLE 9
Lead time estimation of PEB and ST methods at a predefined
1.0% 6-year risk specificity threshold among case participants
stratified by stage and histological subtype.
Adenocarcinoma
Improvement of PEB

| | ST remained | PEB signaled |
|---|---|---|
| | negative | earlier than ST |
| N | 3 | 4 |
| # of Non-USPSTF2013 eligible | 2 | 0 |
| Lead Time of PEB (Years, IQR) | 2.37 (1.75-3.10) | 2.52 (1.89-3.20) |
| Lead Time of ST (Years, IQR) | NA | 0.37 (0.15-0.68) |

Improvement of ST

| | PEB remained | ST signaled |
|---|---|---|
| | negative | earlier than PEB |
| N | 0 | 1 |
| # of Non-USPSTF2013 eligible | 0 | 0 |
| Lead Time of PEB (Years, IQR) | NA | 1.28 |
| Lead Time of ST (Years, IQR) | NA | 2.18 |

Squamous Cell Carcinoma
Improvement of PEB

| | ST remained | PEB signaled |
|---|---|---|
| | negative | earlier than SET |
| N | 6 | 0 |
| # of Non-USPSTF2013 eligible | 1 | 0 |
| Lead Time of PEB (Years, IQR) | 1.66 (1.28-1.73) | NA |
| Lead Time of ST (Years, IQR) | NA | NA |

Improvement of ST

| | PEB remained | ST signaled |
|---|---|---|
| | negative | earlier than PEB |
| N | 0 | 0 |
| # of Non-USPSTF2013 eligible | 0 | 0 |
| Lead Time of PEB (Years, IQR) | NA | NA |
| Lead Time of ST (Years, IQR) | NA | NA |

Small cell lung cancer
Improvement of PEB

| | ST remained | PEB signaled |
|---|---|---|
| | negative | earlier than ST |
| N | 2 | 3 |
| # of Non-USPSTF2013 eligible | 0 | 0 |
| Lead Time of PEB (Years, IQR) | 1.60 (1.0-2.19) | 3.72 (2.88-4.05) |
| Lead Time of ST (Years, IQR) | NA | 2.70 (3.08-1.88) |

Improvement of ST

| | PEB remained | ST signaled |
|---|---|---|
| | negative | earlier than PEB |
| N | 0 | 0 |
| # of Non-USPSTF2013 eligible | 0 | 0 |
| Lead Time of PEB (Years, IQR) | NA | NA |
| Lead Time of ST (Years, IQR) | NA | NA |

Early (I + II) Stage
Improvement of PEB

| | ST remained | PEB signaled |
|---|---|---|
| | negative | earlier than ST |
| N | 8 | 6 |
| # of Non-USPSTF2013 eligible | 3 | 0 |
| Lead Time of PEB (Years, IQR) | 1.66 (1.01-1.83) | 2.03 (1.64-3.30) |
| Lead Time of ST (Years, IQR) | NA | 0.79 (0.24-1.04) |

Improvement of ST

| | PEB remained | ST signaled |
|---|---|---|
| | negative | earlier than PEB |
| N | 0 | 1 |
| # of Non-USPSTF2013 eligible | 0 | 0 |
| Lead Time of PEB (Years, IQR) | NA | 1.28 |
| Lead Time of ST (Years, IQR) | NA | 2.18 |

Late (III + IV) Stage
Improvement of PEB

| | ST remained | PEB signaled |
|---|---|---|
| | negative | earlier than ST |
| N | 6 | 4 |
| # of Non-USPSTF2013 eligible | 2 | 1 |
| Lead Time of PEB (Years, IQR) | 1.70 (1.17-2.31) | 2.88 (2.72-3.20) |
| Lead Time of ST (Years, IQR) | NA | 1.65 (1.21-1.98) |

Improvement of ST

| | PEB remained | ST signaled |
|---|---|---|
| | negative | earlier than PEB |
| N | 0 | 0 |
| # of Non-USPSTF2013 eligible | 0 | 0 |
| Lead Time of PEB (Years, IQR) | NA | NA |
| Lead Time of ST (Years, IQR) | NA | NA |

N represents the number of case participants;
Abbreviations: IQR—interquartile range

TABLE 10
Lead time estimation of PEB and ST methods at a predefined 1.7%
6-year risk specificity threshold among different risk strata.
High risk strata
Improvement of PEB

| | ST remained | PEB signaled |
|---|---|---|
| | negative | earlier than ST |
| N | 17 | 9 |
| # of Non-USPSTF2013 eligible | 0 | 0 |
| Lead Time of PEB (Years, IQR) | 1.36 (1.01-2.05) | 3.03 (2.18-3.72) |
| Lead Time of ST (Years, IQR) | NA | 1.28 (1.02-1.74) |

Improvement of ST

| | PEB remained | ST signaled |
|---|---|---|
| | negative | earlier than PEB |
| N | 0 | 0 |
| # of Non-USPSTF2013 eligible | 0 | 0 |
| Lead Time of PEB (Years, IQR) | NA | NA |
| Lead Time of ST (Years, IQR) | NA | NA |

Medium risk strata
Improvement of PEB

| | ST remained | PEB signaled |
|---|---|---|
| | negative | earlier than ST |
| N | 9 | 2 |
| # of Non-USPSTF2013 eligible | 9 | 2 |
| Lead Time of PEB (Years, IQR) | 1.22 (0.59-1.82) | 3.11 (2.74-3.49) |
| Lead Time of ST (Years, IQR) | NA | 1.03 (0.91-1.15) |

Improvement of ST

| | PEB remained | ST signaled |
|---|---|---|
| | negative | earlier than PEB |
| N | 3 | 2 |
| # of Non-USPSTF2013 eligible | 0 | 0 |
| Lead Time of PEB (Years, IQR) | NA | 0.45 (0.37-0.54) |
| Lead Time of ST (Years, IQR) | 1.02 (0.97-2.11) | 2.39 (2.30-2.49) |

Low risk strata
Improvement of PEB

| | ST remained | PEB signaled |
|---|---|---|
| | negative | earlier than ST |
| N | 2 | 0 |
| # of Non-USPSTF2013 eligible | 2 | 0 |
| Lead Time of PEB (Years, IQR) | 2.68 (1.92-3.42) | NA |
| Lead Time of ST (Years, IQR) | NA | NA |

Improvement of ST

| | PEB remained | ST signaled |
|---|---|---|
| | negative | earlier than PEB |
| N | 1 | 0 |
| # of Non-USPSTF2013 eligible | 1 | 0 |
| Lead Time of PEB (Years, IQR) | NA | NA |
| Lead Time of ST (Years, IQR) | 0.9 | NA |

N represents the number of case participants;
Abbreviations: IQR—interquartile range

TABLE 11
Lead time estimation of PEB and ST methods at a
predefined 1.7% 6-year risk specificity threshold among
case participants stratified by stage and histological subtype.
Adenocarcinoma
Improvement of PEB

| | ST remained | PEB signaled |
|---|---|---|
| | negative | earlier than ST |
| N | 7 | 4 |
| # of Non-USPSTF2013 eligible | 5 | 0 |
| Lead Time of PEB (Years, IQR) | 1.07 (1.0-1.75) | 3.37 (2.60-4.01) |
| Lead Time of ST (Years, IQR) | NA | 0.50 (0.36-1.42) |

Improvement of ST

| | PEB remained | ST signaled |
|---|---|---|
| | negative | earlier than PEB |
| N | 0 | 1 |
| # of Non-USPSTF2013 eligible | 0 | 0 |
| Lead Time of PEB (Years, IQR) | NA | 0.29 |
| Lead Time of ST (Years, IQR) | NA | 2.2 |

Squamous Cell Carcinoma
Improvement of PEB

| | ST remained | PEB signaled |
|---|---|---|
| | negative | earlier than SET |
| N | 11 | 2 |
| # of Non-USPSTF2013 eligible | 3 | 0 |
| Lead Time of PEB (Years, IQR) | 1.19 (0.81-1.66) | 2.80 (2.42-3.17) |
| Lead Time of ST (Years, IQR) | NA | 1.22 (1.12-1.31) |

Improvement of ST

| | PEB remained | ST signaled |
|---|---|---|
| | negative | earlier than PEB |
| N | 0 | 1 |
| # of Non-USPSTF2013 eligible | 0 | 0 |
| Lead Time of PEB (Years, IQR) | NA | 0.62 |
| Lead Time of ST (Years, IQR) | NA | 2.59 |

Small cell lung cancer
Improvement of PEB

| | ST remained | PEB signaled |
|---|---|---|
| | negative | earlier than ST |
| N | 5 | 1 |
| # of Non-USPSTF2013 eligible | 2 | 0 |
| Lead Time of PEB (Years, IQR) | 1.82 (1.05-2.37) | 3.47 |
| Lead Time of ST (Years, IQR) | NA | 1.5 |

Improvement of ST

| | PEB remained | ST signaled |
|---|---|---|
| | negative | earlier than PEB |
| N | 1 | 0 |
| # of Non-USPSTF2013 eligible | 0 | 0 |
| Lead Time of PEB (Years, IQR) | NA | NA |
| Lead Time of ST (Years, IQR) | 1.02 | NA |

Early (I + II) Stage
Improvement of PEB

| | ST remained | PEB signaled |
|---|---|---|
| | negative | earlier than ST |
| N | 16 | 3 |
| # of Non-USPSTF2013 eligible | 7 | 0 |
| Lead Time of PEB (Years, IQR) | 1.03 (0.57-1.38) | 3.72 (3.59-4.31) |
| Lead Time of ST (Years, IQR) | NA | 1.50 (1.03-2.72) |

Improvement of ST

| | PEB remained | ST signaled |
|---|---|---|
| | negative | earlier than PEB |
| N | 1 | 0 |
| # of Non-USPSTF2013 eligible | 0 | 0 |
| Lead Time of PEB (Years, IQR) | NA | NA |
| Lead Time of ST (Years, IQR) | 3.2 | NA |

Late (III + IV) Stage
Improvement of PEB

| | ST remained | PEB signaled |
|---|---|---|
| | negative | earlier than ST |
| N | 13 | 7 |
| # of Non-USPSTF2013 eligible | 7 | 1 |
| Lead Time of PEB (Years, IQR) | 1.82 (1.07-2.16) | 3.03 (2.39-3.39) |
| Lead Time of ST (Years, IQR) | NA | 0.78 (0.31-1.22) |

Improvement of ST

| | PEB remained | ST signaled |
|---|---|---|
| | negative | earlier than PEB |
| N | 1 | 1 |
| # of Non-USPSTF2013 eligible | 0 | 0 |
| Lead Time of PEB (Years, IQR) | NA | 0.62 |
| Lead Time of ST (Years, IQR) | 1.02 | 2.59 |

N represents the number of case participants;
Abbreviations: IQR—interquartile range

TABLE 12
Distribution of positive test results by PEB
and ST methods among non-case participants.

| Test | Number of + Test Results/Non-Case | | | | | Total Non-Case | Total False- |
|---|---|---|---|---|---|---|---|
| method: | 1 | 2 | 3 | 4 | 5 | Participants w/+ Test | Positive |
| Specificity: | | | | | | | |
| 63.2% | | | | | | | |
| PEB | 606 | 489 | 220 | 65 | 3 | 1,383 | 2,521 |
| ST | 236 | 185 | 229 | 146 | 129 | 925 | 2,521 |
| Specificity: | | | | | | | |
| 45.4% | | | | | | | |
| PEB | 387 | 547 | 411 | 204 | 41 | 1,590 | 3,740 |
| ST | 219 | 226 | 323 | 230 | 235 | 1,235 | 3,740 |

Serial measurement of 4MP within the context of an adaptive PEB algorithm model improves sensitivity and lead time for risk assessment of lung cancer for lung cancer screening compared to a ST approach. Testing of the 4MP would be useful for individuals who are currently eligible for LDCT screening in addition to expansion to those with a ≥10 PY smoking history for risk-based referral to LDCT screening through shared decision making. Individuals with an initial ‘negative’ test would be recommended for repeat testing with testing intervals matching their degree of risk. This creates a scenario whereby information from repeat measurements may provide additional information regarding lung cancer risk.

The advantage of PEB is the ability to adjust for low values of the 4MP at baseline whereby incremental increases in the 4MP can identify those at risk of lung cancer despite being ST negative. To this end, Volume Doubling Time (VDT), defined as the number of days in which a nodule doubles its volume, is a clinically important metric in lung cancer screening with a general range of 20 days to <590 days for malignant nodules. Changes in biomarker levels may reflect dissemination from an occult tumor into blood during cancer proliferation and progression. In this example, at the 1% 6-year risk threshold, the PEB algorithm led to an earlier positive ‘test’ in 43% of patients that were subsequently diagnosed with lung cancer, with an average lead time of 1.37 years, which is anticipated to result in a stage-shift and mortality reduction benefit.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A method of determining the risk of a patient for having a lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as being at risk for lung cancer or not being at risk for lung cancer by comparing the model score to a pre-defined parameter.

2. A method of improving lead time prior to diagnosis of a patient having lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point; and
identifying the patient as having an increased risk of lung cancer by comparing the model score to a pre-defined parameter.

3. A method comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point;
identifying the patient as being at risk for lung cancer or not being at risk for lung cancer by comparing the model score to a pre-defined parameter; and
administering a computerized tomography (CT) scan to the patient identified as being at risk for lung cancer.

4. A method of identifying and treating an asymptomatic patient with a risk for increased lung cancer, comprising:
calculating a model score using two or more biomarker scores collected at two or more time points, wherein each biomarker score is determined from the levels of biomarkers CEA, CA125, CYFRA21-1, and Pro-SFTPB in a biological sample obtained from the patient at each time point;
identifying the patient as being at risk for lung cancer or not being at risk for lung cancer by comparing the model score to a pre-defined parameter;
administering a computerized tomography (CT) scan to the patient identified as being at risk for lung cancer; and
surgically removing the cancerous tumors identified on the CT scan.

5. The method of claim 4, wherein the model score is calculated with the equation:
$$Model Score = \frac{Y - [ μ \times ( 1 - {B}_{n} ) + {\overline{X}}_{i} \times {B}_{n} ]}{\sqrt{V \times ( 1 - {B}_{1} \times {B}_{n} )}}$$
wherein:
Y is the biomarker score of the most recent time point and is calculated with the equation
$$0.473 * log [ CA 125 ] + 0.6531 * log [ CEA ] + 0.2612 * log [ CYFRA 21 - 1 ] + 0.9238 * log [ Pro - SFTPB ] ;$$
μ is about 7.07;
V is about 0.2672;
B₁ is about 0.767;
Bₙ is calculated with the equation (n*B₁)/(n*B₁+(1−B₁)), wherein n is the total number of tests performed on the patient; and
X̅ᵢ is the average of Y scores calculated from biomarkers collected at time points prior to the most recent time point.

6. The method of claim 1, wherein the pre-defined parameter is about 0.63.

7. The method of claim 1, wherein the pre-defined parameter is about 0.45.

8. The method of claim 1, wherein a model score greater than the pre-defined parameter is considered a positive test.

9. (canceled)

10. The method of claim 1, wherein the biomarker scores are collected at between two and five time points.

11-20. (canceled)

21. The method of claim 1, wherein the lung cancer is stage I or II.

22. The method of claim 1, wherein the lung cancer is stage III or IV.

23. The method of claim 1, wherein the individual has a smoking history of ≥10 pack years.

24. The method of claim 1, wherein the individual is between the age of 50 and 80 years.

25. (canceled)

26. The method of claim 2, wherein the lead time to diagnosis is greater than 1.03 years.

27. The method of claim 26, wherein the lead time to diagnosis is between 1.26 and 2.70 years.

28. The method of claim 1, wherein the patient is subsequently administered further lung cancer screening or treatment.

29. The method of claim 28, wherein the screening is chosen from endoscopic ultrasound, magnetic resonance imaging (MRI), and computed topography (CT) scans.

30. The method of claim 29, wherein the screening is performed annually.

31. The method of claim 29, wherein the screening is performed semi-annually.

32. The method of claim 28, wherein the treatment is chosen from surgery, chemotherapy, immunotherapy, radiation therapy, targeted therapy, or a combination thereof.
